(19) United States
(12) Patent Application Publication
(10) Pub. No.: US 2021/0366602 A1
(43) Pub. Date: Nov. 25, 2021

(54) SIGNAL-PROCESSING DEVICE, ANALYSIS SYSTEM, SIGNAL-PROCESSING METHOD, AND SIGNAL-PROCESSING PROGRAM

(71) Applicant: NEC Corporation, Minato-ku, Tokyo (JP)

(72) Inventors: Chenhui HUANG, Tokyo (JP); Jingwen LU, Tokyo (JP)

(73) Assignee: NEC Corporation, Minato-ku, Tokyo (JP)

(21) Appl. No.: 16/957,261

(22) PCT Filed: Nov. 7, 2018
(86) PCT No.: PCT/JP2018/041337

§ 371 Date: Jun. 23, 2020

(30) Foreign Application Priority Data
Dec. 28, 2017 (JP) 2017-253457

(51) Int. Cl. G16H 40/63; A61B 5/397

(52) U.S. Cl. CPC G16H 40/63; A61B 5/397

(57) ABSTRACT

Even when series data is acquired under an acquisition condition in which a part of valid components is not included, significant information is acquired from the series data highly accurately. A signal-processing device 600 of the present invention includes: a data acquisition unit 601 that acquires series data or data included therein; and a data-processing unit 602 that generates variable band data that is data related to a bandwidth of a variable band expressed by a predetermined constant multiple in a positive/negative direction of a variability parameter that is a parameter acquired by application of a time window of a predetermined time length to target series data, which is the acquired series data or series data including the acquired data, after a removal of a moving average and that is a parameter indicating variability of the target series data in the time window.

### TECHNICAL FIELD

The present invention relates to a signal-processing device for processing series data, an analysis system using the same, a signal-processing method, and a signal-processing program.

### BACKGROUND ART

A signal-processing device that processes series data is used for many purposes such as a purpose of estimating or determining a current state of an observation target such as a space, person, or object from which the series data is observed, or predicting a future state thereof on the basis of significant information such as a feature associated with a change in the series data which information is acquired by an analysis of the series data.

Recently, downsizing of such a signal-processing device is in progress. There is a case where accuracy of a data acquisition mechanism is decreased, a signal-processing capacity is decreased, or an amount of data that can be acquired is decreased along with downsizing of the device. Even in such a case, it is required to extract meaningful information with high accuracy from acquired series data.

As an example of the series data, there is time-series data of a myoelectric signal shown in electromyography (EMG). A muscular substance includes many muscle fibers, and action potential (muscle potential) generated by excitation of the muscle fibers which excitation is associated with contraction of the muscular substance is measured and visualized in the electromyography. The muscle potential is potential (small change in electric field) generated by related muscle fibers when the muscular substance contracts. An electric signal by this potential observed via an electrode or the like is generally called a myoelectric signal or a muscle potential signal. By measuring and decoding such a myoelectric signal, activity of muscle can be grasped quantitatively.

An example of an integration method is described in PTL 1 as a signal-processing method for measuring a muscle activity amount that is an amount of muscle activity. In the signal-processing method according to PTL 1, with respect to myoelectric signals that are input every moment, an integral quantity in a certain time length is calculated after rectification is performed. Here, the rectification means to acquire an absolute value of a myoelectric signal after making the signal pass through a direct-current filter (DC filter). The rectified myoelectric signal is integrated in a certain time length, and the integral quantity is acquired as a muscle activity amount. The muscle activity amount is one piece of meaningful information acquired from time-series data of the myoelectric signal.

### CITATION LIST

### Patent Literature

PTL 1: Japanese Patent Application Laid-Open No. 2008-054955

### SUMMARY OF INVENTION

### Technical Problem

Muscle potential (small change in electric field which change is based on command from brain) that appears in a myoelectric signal generally lasts only for a short period (about 1 second), and the change (rise or fall of signal) is steep. When a sampling rate for a myoelectric signal is high, a shape of an original myoelectric signal (electrical signal accurately expressing muscle potential generated in muscle of observation source) also is kept well in time-series data of acquired myoelectric signal. Thus, a valid component (specifically, valid component indicating feature in time change) does not fall out, and evaluation accuracy of a muscle activity amount is not decreased.

However, in a case where a signal acquisition mechanism (such as sensor) is mounted in a wearable device or the like, there is a case where a sampling rate of the sensor needs to be reduced for a purpose of power saving, or the like. When a sampling rate for a myoelectric signal is reduced, a part of valid components is lost from time-series data of an acquired myoelectric signal. It is not possible to acquire an accurate muscle activity amount by simply performing rectification and applying an integration method with respect to time-series data of such a myoelectric signal.

Note that such a problem is not limited to a myoelectric signal, and is generated similarly in series data acquired with a part of valid components being lost from a significant change form of an original signal due to a shortage in a sampling rate, or the like.

Thus, the present invention is to provide a signal-processing device, a signal-processing method, and a signal-processing program that are capable of acquiring significant information from series data highly accurately even when the series data is acquired under an acquisition condition in which a part of valid components is not included. Also, the present invention is to provide an analysis system capable of analyzing a state of an observation target of series data highly accurately even when the series data is acquired under an acquisition condition in which a part of valid components is not included.

### Solution to Problem

A signal-processing device according to the present invention includes: a data acquisition unit that acquires series data or data included therein; and a data-processing unit that generates variable band data that is data related to a bandwidth of a variable band expressed by a predetermined constant multiple in a positive/negative direction of a variability parameter that is a parameter acquired by application of a time window of a predetermined time length to target series data, which is the acquired series data or series data including the acquired data, after a removal of a moving average and that is a parameter indicating variability of the target series data in the time window.

An analysis system according to the present invention includes: a signal collection unit that collects a signal at a predetermined sampling rate; the above-described signal-processing device in which data included in series data is the signal collected by the signal collection unit; and a state estimation unit that estimates a state of an observation source of the series data on the basis of information acquired by the signal-processing device.

A signal-processing method according to the present invention includes generating, when acquiring series data or data included therein, variable band data that is data related to a bandwidth of a variable band expressed by a predetermined constant multiple in a positive/negative direction of a variability parameter that is a parameter acquired by application of a time window of a predetermined time length to target series data, which is the acquired series data or series data including the acquired data, after a removal of a moving average and that is a parameter indicating variability of the target series data in the time window, the generating being performed by an information-processing device.

A signal-processing program according to the present invention causes a computer to execute processing of acquiring series data or data included therein, and processing of generating variable band data that is data related to a bandwidth of a variable band expressed by a predetermined constant multiple in a positive/negative direction of a variability parameter that is a parameter acquired by application of a time window of a predetermined time length to target series data, which is the acquired series data or series data including the acquired data, after a removal of a moving average and that is a parameter indicating variability of the target series data in the time window.

### Advantageous Effects of Invention

According to the present invention, even when series data is acquired under an acquisition condition in which a part of valid components is lost, significant information can be acquired from the series data highly accurately. Also, a state of an observation target of such series data can be analyzed highly accurately.

### BRIEF DESCRIPTION OF DRAWINGS

FIG. 1 It depicts a block diagram illustrating a configuration example of a signal-processing device 10 of a first exemplary embodiment.

FIG. 2 It depicts a block diagram illustrating a more detailed configuration example of a signal-processing unit 12.

FIG. 3 It depicts a flowchart illustrating an operation example of the signal-processing device 10 of the first exemplary embodiment.

FIG. 4 It depicts a flowchart illustrating an example of a more detailed operation of the signal-processing device 10.

FIG. 5 It depicts a graph illustrating an example of myoelectric signal data W and a baseline B.

FIG. 6 It depicts a graph illustrating an example of a variability parameter curve Σ.

FIG. 7 It depicts a graph illustrating an example of myoelectric signal data W (100 Hz) and a band (B±k*Σ) acquired therefrom.

FIG. 8 It depicts a view for describing an example of an effect of a band area method.

FIG. 9 It depicts a graph illustrating an example of myoelectric signal data W (2 Hz) and a band (B±k*Σ) acquired therefrom.

FIG. 10 It depicts a block diagram illustrating a configuration example of a signal-processing device 20 of a second exemplary embodiment.

FIG. 11 It depicts a flowchart illustrating an operation example of the signal-processing device 20 of the second exemplary embodiment.

FIG. 12 It depicts a block diagram illustrating a configuration example of an analysis system of a third exemplary embodiment.

FIG. 13 It depicts a block diagram of a signal-processing unit 12A in more detail.

FIG. 14 It depicts a schematic block diagram illustrating a configuration example of a computer according to each exemplary embodiment of the present invention.

FIG. 15 It depicts a block diagram illustrating an outline of a signal-processing device of the present invention.

### DESCRIPTION OF EMBODIMENTS

### First Exemplary Embodiment

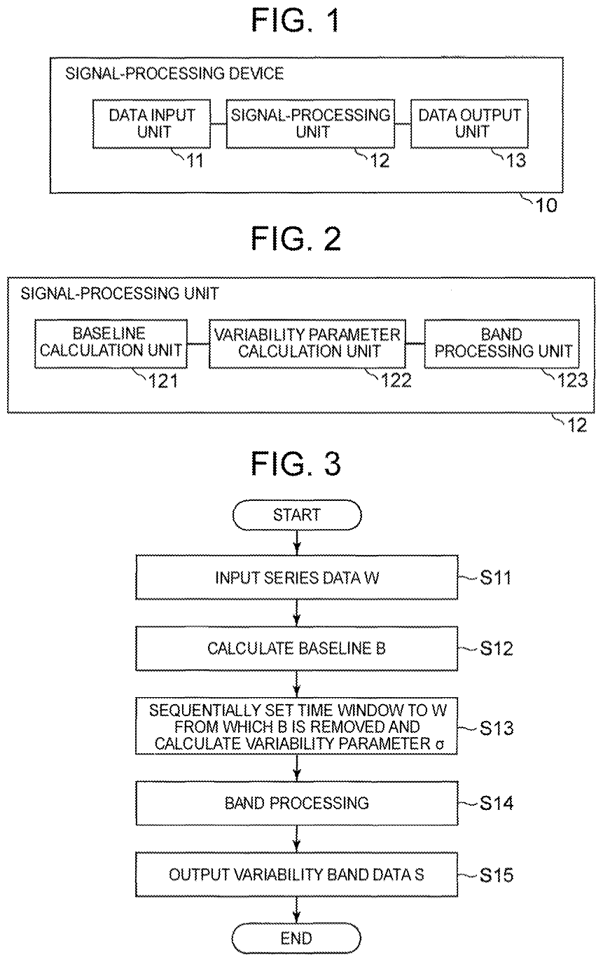

In the following, exemplary embodiments of the present invention will be described with reference to the drawings. FIG. 1 is a block diagram illustrating a configuration example of a signal-processing device 10 of the first exemplary embodiment. The signal-processing device 10 illustrated in FIG. 1 includes a data input unit 11, a signal-processing unit 12, and a data output unit 13.

The data input unit 11 inputs series data or each piece of data included therein. For example, the data input unit 11 may be a data input device that inputs a signal to be observed from the outside and outputs an input signal as series data to a signal-processing unit 12 in a following stage while buffering the signal for a certain amount. When performing the output to the signal-processing unit 12 in the following stage, the data input unit 11 may sequentially output input signals or may output series data including a predetermined amount of elements.

In the following, a case where series data is time-series data of a signal acquired by a predetermined sensor or the like will be described as an example. However, the series data is not limited to time-series data of a signal. For example, a value of the series data may change according to an increase in the number of times of a certain action or measure, or the like. In other words, a data set in which pieces of data are arranged with order thereof being kept on a predetermined axis expressing timing of acquisition or generation is regarded as series data of the present invention. In that case, an index i corresponding to time, a time window, or the like is replaced with an index indicating an arbitrary time point on an axis on which data such as the number of times is arranged, a window having a predetermined length on the axis, or the like.

Also, with respect to one piece of data (signal) w, series data as an aggregate thereof may be referred to as W or W( ) in the following. Furthermore, there is a case where certain data included in the series data and expressed by an index i corresponding to time is referred to as W(i). Here, i is an index indicating arbitrary data w included in series data to be processed, and may be also used as information indicating a distance (relative time) from a reference time point on a time axis of the series data. Note that the above notation (however, with different symbol) is also used for series data including data other than w. That is, with respect to data expressed by a symbol in lowercase, series data that is an aggregate thereof is expressed by the symbol in uppercase, and certain data therein is expressed with an index enclosed in ( ) when indicated.

When series data or each piece of data included therein is input, the signal-processing unit 12 performs predetermined processing thereon and generates variable band data that is data, which indicates a bandwidth in each period of a variable band that is a band corresponding to a changeable space of the series data indicated by the input data, or series data thereof. Each piece of data included in this variable band data is information indicating a total amount of signals in a time length corresponding to an interval of acquisition of series data to be processed. In the following, the series data to be processed is referred to as W, each piece of data included therein is referred to as w, variable band data is referred to as S, and each piece of data included in the variable band data is referred to as s.

More specifically, the data s and the variable band data S are data indicating a bandwidth of a variable band indicated by a predetermined constant multiple in a positive/negative direction of a variability parameter, and series data thereof. The variability parameter is a parameter acquired by application of a time window of a predetermined time length to the series data W, which is indicated by the input data, after a removal of a baseline B expressed by a moving average, and is a parameter that is associated with an index set as a reference of the time window and that indicates variability (such as dispersion) of the series data W in the time window.

In association with an index i of the series data W indicated by the input data, the data output unit 13 outputs the data s or the variable band data S that is series data thereof, the data being acquired by the signal-processing unit 12.

FIG. 2 is a block diagram illustrating a configuration example of the signal-processing unit 12 in more detail. As illustrated in FIG. 2, the signal-processing unit 12 includes a baseline calculation unit 121, a variability parameter calculation unit 122, and a band processing unit 123.

The baseline calculation unit 121 calculates a baseline B of series data W to be processed.

The variability parameter calculation unit 122 sequentially applies a predetermined time window TW to the series data W, and calculates a variability parameter σ expressing variability of a variation D of the series data W with respect to the baseline B in the time window TW.

The band processing unit 123 performs band processing on a variability parameter curve Σ acquired by arrangement of calculated variability parameters σ in time order, forms (calculate) a band expressing a variable space of the series data W (variable band), and generates variable band data S.

Next, an operation in the present exemplary embodiment will be described. FIG. 3 is a flowchart illustrating an operation example of the signal-processing device 10 of the present exemplary embodiment. In the example illustrated in FIG. 3, first, the data input unit 11 inputs series data W to be processed (Step S11). Here, the series data W only needs to include at least two pieces of data w. Note that pieces of data w may be transferred, as series data W to be processed, to a processing unit in a following stage by being sequentially input and buffered for a predetermined amount.

When the series data W is input by the data input unit 11, the baseline calculation unit 121 calculates a baseline B (Step S12).

Next, after removing the baseline B from the series data W, the variability parameter calculation unit 122 sequentially applies a predetermined time window to the series data W, and calculates a variability parameter σᵢ=Σ(i) associated with an index i of the series data W (Step S13).

Next, the band processing unit 123 performs band processing on a variability parameter curve Σ expressed by the variability parameter σᵢ (Step S14). In Step S14, the band processing unit 123 performs the band processing on the variability parameter curve Σ, calculates a variable band expressing a variable space of the series data W, and acquires variable band data S.

The data output unit 13 outputs the acquired variable band data S (Step S15).

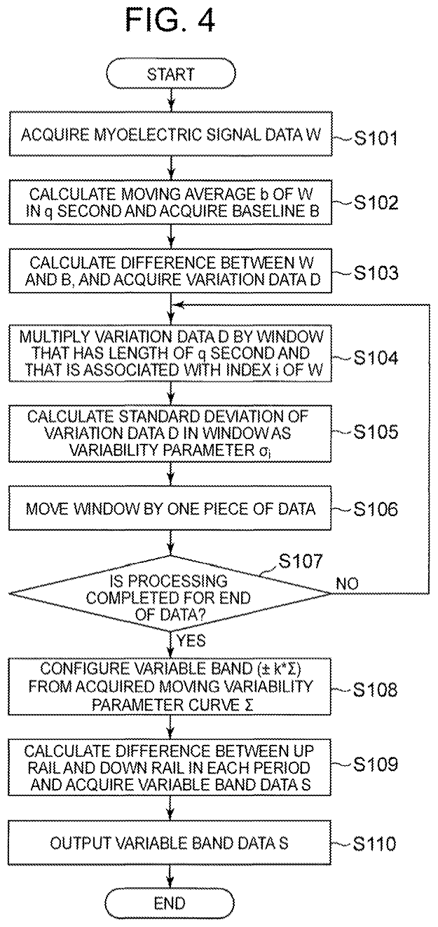

Next, the above-described each kind of processing will be described in more detail with reference to FIG. 4. FIG. 4 is a flowchart illustrating an example of a more detailed operation of the signal-processing device 10 of the present exemplary embodiment. Note that a case where myoelectric signal data, which is time-series data of myoelectric signals, is input as series data W will be described as an example in FIG. 4.

The myoelectric signals are signals indicating potential (muscle potential) generated during muscle activity, as described above. This muscle potential is generated along a muscle fiber that is a muscle cell. Such myoelectric signals can be measured, for example, by an electrode attached to a surface of muscle. Also, there are not only a method of directly performing acquisition via an electrode but also a method of performing acquisition by remote measurement such as estimating a myoelectric signal from a muscle activity image acquired by photographing of activity of muscle with a camera device. Note that a method of acquiring myoelectric signal data does not matter in the present exemplary embodiment.

Unlike a pulse wave signal or an electrocardiographic signal, a myoelectric signal does not have a clear periodic feature, and has a feature of an incidental signal that reflects instantaneous movement of muscle. Thus, generally, only a variation in a time domain needs to be considered in an analysis, and a frequency component does not need to be considered unlike the pulse wave signal or the electrocardiographic signal. That is, only the variation in the time domain is an object of the analysis with respect to the myoelectric signal.

The signal-processing unit 12 of the present exemplary embodiment has a function of generating a signal capable of stochastically expressing a change tendency from a signal even when the signal is collected at a low sampling rate.

When a signal is simply considered as a combination of signals of different frequencies, in a case where a sampling rate becomes lower than a Nyquist frequency of a valid component of the signal, the valid component cannot be measured accurately. However, when a signal that does not have a clear periodic feature, an incidental signal, or the like is considered as data having a statistical property in a change of signal intensity over time (such as Gaussian distribution), data at time n+1 can be expressed by a conditional probability of data at time n. This means that a lost component and a change tendency of a signal can be stochastically expressed even when a sampling rate for the signal is low. When the change tendency of the signal is known stochastically, even in a case where a true value of the signal cannot be defined, an existence range thereof can be specified. By utilizing the existence range of the signal which range is specified in such a manner, it is possible to express a feature of whole data.

As illustrated in FIG. 4, in the present example, first, myoelectric signal data (series data W) including a predetermined number of myoelectric signals (data w) or more is input (Step S101). Here, the input myoelectric signal data is myoelectric signal data (EMG data) passing through a DC filter.

Next, the baseline calculation unit 121 calculates a baseline of the myoelectric signal data (Step S102).

As already described, muscle potential appears when muscle activity is generated, and the potential lasts for about q seconds (q is generally 1 to 2 seconds). Thus, by calculating a moving average bᵢ=B(i) in an interval of Δt=q seconds corresponding to an index i of the myoelectric signal data passing through the DC filter, the baseline calculation unit 121 can express a baseline B by a set thereof.

Equation (1) in the following is an example of an equation for calculating a moving average bᵢ in a time interval TSᵢ corresponding to the index i of the series data W. Equation (1) is an example of calculating a moving average bᵢ of a case where data included in the time interval TSᵢ corresponding to the index i of the series data W is signals W(i−(n−1)) to W(i).

$$\begin{matrix}[ Math 1 ] & \\ B ( i ) = {b}_{i} = \frac{1}{n} {\sum }_{k = 0}^{n - 1} W ( i - k ) & ( 1 )\end{matrix}$$

Here, n is the number of pieces of data w included in the time interval TS for Δt seconds (myoelectric signal as EMG signals included in EMG data). Also, a subscript i of a moving average b indicates that the value is a value in the time interval TSᵢ set in association with the index i of the series data W (myoelectric signal data, that is, EMG data).

Note that as expressed in equation (1), when the moving average bᵢ is used as it is as a baseline value B(i) of the baseline B, a delay for 0.5*q seconds is generated in B with respect to W. Thus, when W and B are placed on the same time axis, a coincidence of the data on the time axis is kept by W being delayed for 0.5*q seconds or B being advanced for 0.5*q seconds. More specifically, the above time interval TS is advanced for n/2 pieces of data, that is, an index value in W( ) on a right side of equation (1) is changed to (i−n/2−k), (i−(n+1)/2−k), or the like.

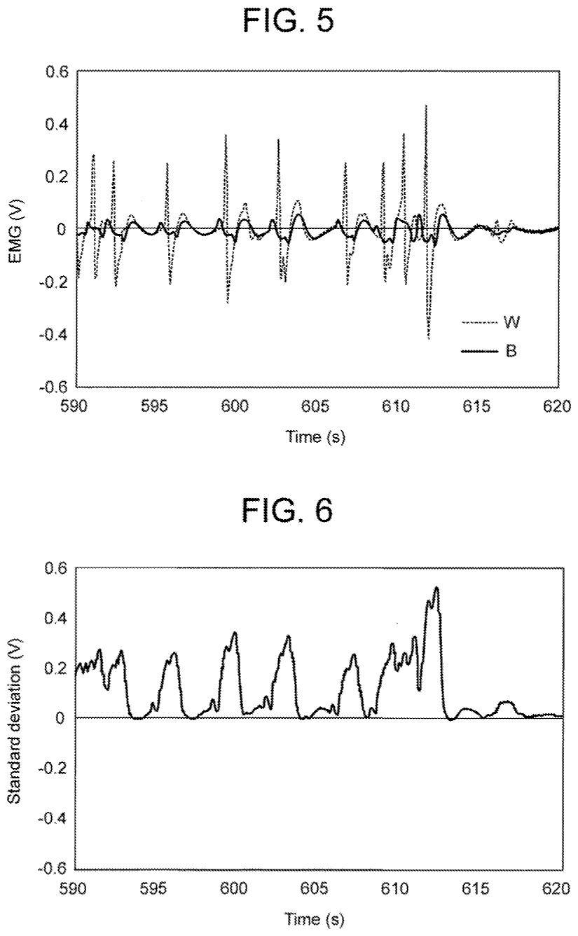

FIG. 5 is a graph illustrating an example of the series data W and the baseline B. In the example illustrated in FIG. 5, the moving average bᵢ is calculated with respect to the series data W (myoelectric signal data) while an index i to which the time interval TS is applied is incremented by one, and a set thereof is set as the baseline B. Note that in FIG. 5, data of the two is displayed after time axes thereof are made to match. As illustrated in FIG. 5, a fluctuation of W can be expressed by the baseline B when a moving average in a time interval according to a signal (muscle potential) to be detected is calculated. Such a fluctuation of the series data W is often a noise due to a condition in measurement, or a noise due to a property of a signal collection function or an individual to be observed (such as noise generated by body movement or contact condition of electric circuit, or inherent noise of semiconductor device included in circuit) and is not a time change of a signal to be originally detected in many cases.

When the baseline B is acquired, the variability parameter calculation unit 122 subsequently calculates, for each piece of data in the series data W, a variation d=D(i) with respect to the baseline B (Step S103). Then, the variability parameter calculation unit 122 calculates a variability parameter σᵢ associated with i of W by using variation data D that is series data of the variations D(i) (Step S104 to Step S107).

In the present example, a time window TWᵢ having a time length of Δt=q seconds is sequentially applied to the variation data D that is the series data of the variations d, and a standard deviation of the variations d in the time window TWᵢ is calculated as a variability parameter σᵢ=Σ(i).

Equation (2) in the following is an example of an equation for calculating, as the variability parameter σᵢ, a standard deviation in the time window TWᵢ corresponding to the index i of the series data W. Equation (2) is an example of calculating a standard deviation of a case where indexes of data w included in the time window TWᵢ of the time length Δt which indexes correspond to the index i of the series data W are i−(n−1) to i.

$$\begin{matrix}[ Math 2 ] & \\ \sum ( i ) = {σ}_{i} = \sqrt{\frac{1}{n} {\sum }_{k = 0}^{n - 1} {D ( i - k )}^{2}} = \sqrt{\frac{1}{n} {\sum }_{k = 0}^{n - 1} {[ W ( i - k ) - B ( i - k ) ]}^{2}} & ( 2 )\end{matrix}$$

The variability parameter σᵢ is not limited to a standard deviation of variations d=D(i) in a time window TWᵢ of a predetermined time length, and may be, for example, a value that is a half or median of a distance from a maximum value wₘₐₓ of raw data (data w) in the time window TWᵢ, or a certain numerical value above/below a moving average b=B(i) in the time window TWᵢ.

For example, when it is assumed that the maximum value of the data w in the time window TWᵢ is wₘₐₓ, a distance L(i) from the maximum value wₘₐₓ to i is calculated by L(i)=wₘₐₓ−D(i). The variability parameter σᵢ may be a value that is a half of this distance L(i), that is, σᵢ=L(i)/2, or may be a median, that is, σᵢ=D(i)+L(i)/2). Also, for example, on the basis of a moving average b(i) in the time window TWᵢ, σᵢ=b(i)+Aα(i), σᵢ=b(i)−Bα(i), or the like may be used. Here, α(i) may be, for example, a distance L(i)=bₘₐₓ−b(i) from a maximum value bₘₐₓ of a moving average b in time TWᵢ in i. Note that A and B are arbitrary constants.

The variability parameter σ is a scalar value. When one σ is calculated, a window for cutting data is moved on a time axis by one piece of data and a next variability parameter σ is calculated. When variability parameters σ are plotted in time order, a variability parameter curve Σ is acquired.

FIG. 6 is a graph illustrating an example of the variability parameter curve Σ. The example illustrated in FIG. 6 is an example in which a moving standard deviation is used as a variability parameter. Here, “moving” indicates that a parameter is acquired by movement of a window for cutting data by one data length in time order. More specifically, it is indicated that the parameter is associated with each of indexes i of the series data W and is calculated by utilization of a data group (w, b, and d) in a predetermined time window TWᵢ including i.

When the variability parameter curve Σ is acquired, the band processing unit 123 subsequently performs band processing on the variability parameter curve Σ and generates variable band data S. When a variability parameter σᵢ=Σ(i) that is each piece of data of the variability parameter curve Σ is multiplied by a constant k, one band (hereinafter, referred to as variable band) is formed between ±k*Σ. This band expresses a variable space of the series data W (in present example, myoelectric signal data). All pieces of data w (myoelectric signal) fall within the band of B±k*Σ with a certain probability according to k (such as 95.4% when k=2, and 99.8% when k=3). Note that a coincidence of data on a time axis is also kept when the variable band is formed. For example, a position at a half of a window length in B or Σ may be set as a reference position of data plotting.

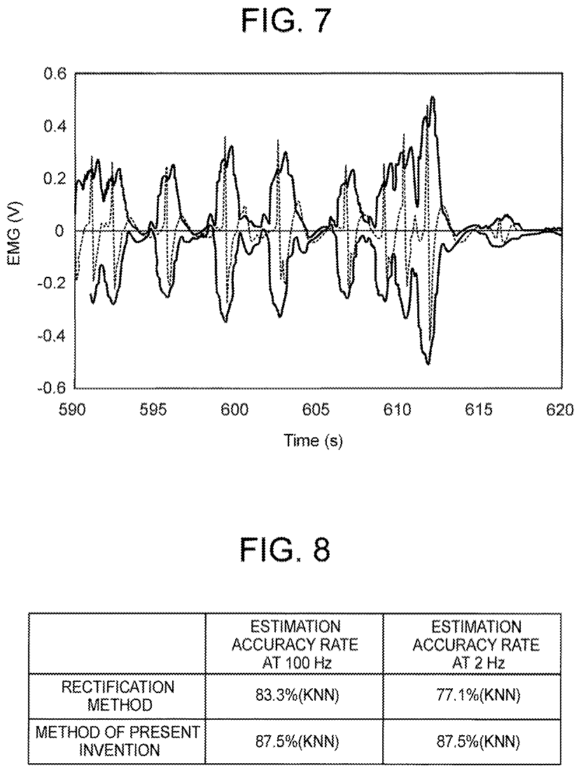

FIG. 7 is a graph illustrating an example of myoelectric signal data W and a band (B±k*Σ) acquired therefrom. Note that the myoelectric signal data W illustrated in FIG. 7 is acquired at a sampling rate of 100 Hz. A broken line is W and a solid line is a band. Also in FIG. 7, data of the two is displayed after time axes thereof are made to match. As illustrated in FIG. 7, the myoelectric signal data W falls within the band (B±k*Σ) when a difference in a vertical direction is ignored, and this band looks like an envelope of W. A feature of the data can be expressed by this band. When a difference S(i)=2k*Σ(i) between an up rail (B+k*Σ rail) and a down rail (B−k*Σ rail) of this band is acquired in the time axis, a muscle activity signal can be expressed by each S(i). With respect to the variability parameter curve Σ, the band processing unit 123 performs such band processing, that is, calculates S(i)=2k*Σ(i) for each i of W (however, limited to what having defined Σ(i)) and acquires variable band data S.

Finally, the data output unit 13 outputs the acquired variable band data S (Step S110).

Note that in FIG. 4, an example of (1) calculating a baseband B, subsequently (2) calculating a variability parameter Σ(i) by sequentially setting a time window TWᵢ for series data W on the basis of a variation D of W with respect to B, and then (3) calculating variable band data S from a variability parameter curve Σ is described with respect to a certain amount of series data W. However, the above processing (1) to (3) can be sequentially performed with respect to pieces of data w input sequentially.

In that case, a buffer or the like that holds at least data w, a moving average b, and a variation d in a time length in which a moving average b and a variability parameter σ can be calculated while coincidence on a time axis is kept with respect to i to be processed is used.

Then, when data W(i) is input, (1) a moving average b in a time interval TS_{i′} of i′ at a center of n pieces of data input so far is calculated by utilization thereof, and is held as a baseline value B(i′). Then, (2) a variation D(i′) of W(i′) with respect to B(i′) is calculated from held W(i′). Then, (3) when a time window TW_{i″} including the variation D(i′) can be set, a time window TW_{i″} is set for i″ at a center thereof, and a variability parameter Σ(i″) is calculated on the basis of a data group (w, b, and d) in the time window TW_{i″}. Finally, (4) the acquired Σ(i″) is multiplied by a predetermined constant (2k) and data S(i″) of the variable band data S is acquired.

For example, it is assumed that data W(i) is input sequentially starting from i=0. Here, it is assumed that time lengths of a time interval for calculating a moving average and a time window for calculating a variability parameter are three data lengths (that is, n=3). In that case, the above processing (1) and (2) is performed from the time of i=2 at which n pieces of data W are held. Here, i′=i−(n−1)/2. Then, at the time of i=4 at which two more pieces of data W are input and n variations D are held, (3) described above is performed and data S(2) of variable band data S is output. Here, i″=i′−(n−1)/2.

An operation for each i is summarized below.
When i=2,

(1) a moving average b is calculated by utilization of W(0) to W(2) and is held as B(1), and

(2) a variation d of W(1) with respect to B(1) is calculated and is held as D(1).
When i=3,

(1) a moving average b is calculated by utilization of W(1) to W(3) and is held as B(2), and

(2) a variation d of W(2) with respect to B(2) is calculated and is held as D(2).
When i=4,

(1) a moving average b is calculated by utilization of W(2) to W(4) and is held as B(3),

(2) a variation d of W(3) with respect to B(3) is calculated and is held as D(3), and

(3) a standard deviation σ of D is calculated by utilization of D(1) to D(3) and is set as Σ(2), and S(2)=2k+Σ(2) is calculated and output.

The above processing (1) to (3) can be also described as follows.

(1) A moving average b is calculated by utilization of W(i−(n−1)) to W(i) and is held as B(i′).

(2) A variation d of W(i′) with respect to B(i′) is calculated and held as D(i′).

(3) A standard deviation σ is calculated by utilization of D(i′−(n−1)) to D(i′) and is set as Σ(i″), and S(i″)=2k+Σ(i″) is calculated and output.

Note that in addition to the above method, for example, it is also possible to perform processing of sequentially applying, after calculating a baseline B for all pieces of data and removing B from W, a time window TWᵢ to the series data W from which B is removed, and calculating a variability parameter σᵢ=Σ(i) and outputting data S(i)=2k*Σ(i) each time one time window TWᵢ is applied.

Generally, a signal (in present example, myoelectric signal) acquired with a measurement device or the like includes noise. The noise is, for example, an electric noise, 1/f noise, noise due to displacement of an electrode in attachment thereof or displacement of an electrode due to a body movement, or the like. Such noise exists as inherent noise of a detection mechanism, and magnitude thereof exists naturally and does not change unless an attribute of a component or the like of the detection mechanism is changed.

Many of conventional signal-processing methods focus only on a peak. Also, even when a peak is detected, isolation between a signal peak and a noise peak is ambiguous. On the other hand, a band acquired by the above method (hereinafter, referred to as band area method) stochastically expresses a tendency of a change in series data W. In this band, an average value of a noise signal is close to 0, and a change rate of an area thereof is close to 0. In addition, in a part where a signal (muscle potential) to be observed exists, a change is drastic, and a stochastic existence section of each piece of data is also increased from an integral after the conventional rectification. Thus, isolation between a signal and noise becomes clearer, an S/N ratio is increased, and a contrast of a change in a feature amount expressed by an integral quantity of data is also increased.

Thus, when variable band data S acquired by the present exemplary embodiment is integrated in a certain time length, that is, when an area of S in a certain time length is acquired, a new feature amount (in above example, new muscle activity amount) can be expressed. The new feature amount is different in a scale from a feature amount acquired by integration of input series data W in a certain time length, but is more accurate.

FIG. 8 is a view for describing an example of an effect of the band area method. In FIG. 8, prediction accuracy rates of a mental state of a person by utilization of a muscle activity amount acquired from myoelectric signal data W at two sampling rates are illustrated for a conventional rectification method and the signal-processing method according to the present invention in comparison with each other. The prediction accuracy rate is a concordance rate between a true value known in advance and an estimation result in a model learned by machine learning.

A mental state of a human appears in mimic muscles, and causes minute changes in muscles around eyes, eyebrows, a mouth, and the like. For example, when the mental state changes, movements such as blinking, eyeball movements, glancing up, and narrowing eyes is generated. In such a manner, a mental state of a person has relevance to activity of muscle, and the mental state can be estimated from a signal of the muscle on the basis of the relevance.

The example illustrated in FIG. 8 is a result of validation, by a cross-validation method, of an accuracy rate of a mental state by a prediction model in which a relationship between a known mental state and each of muscle activity amounts acquired by two methods is machine-learned. Since there is a causal relationship between accuracy of a muscle activity amount and a prediction accuracy rate, the prediction accuracy rate of the mental state becomes higher as the accuracy of the muscle activity amount becomes higher.

First, myoelectric signal data W including a concentration state and a non-concentration state (relaxed state) is acquired from a plurality of subjects. Then, the acquired myoelectric signal data W is divided into learning data and validation data at a ratio of 87.5% and 12.5%. Then, for the acquired myoelectric signal data W, a moving standard deviation is calculated by the above method, and variable band data S by a band thereof is acquired. Furthermore, in the present example, the myoelectric signal data W is acquired at two sampling rates in order to examine a change in the accuracy rate due to a difference in the sampling rates.

From the acquired variable band data S, a bandwidth (data s) of a variable band (±k*Σ) is integrated over time as a muscle activity amount specifically in a time period specifically considered to be in a relaxed state, and an area thereof is calculated. Then, the calculated area is divided by a length of the time period, and an acquired unit time area of the variable band is defined as a unit time muscle activity amount in the relaxed state. Similarly, from the acquired variable band data S, a unit time area of a variable band in a time period specifically considered to be in a concentration state is calculated and defined as a unit time muscle activity amount in the concentration state.

Also, rectification is performed on the myoelectric signal data W from which the above variable band data S is acquired, a muscle activity amount with respect to a similar time period (integral value of signal in the time period) is calculated by a conventional method, the calculated muscle activity amount is divided by a length of the time period, and a unit time muscle activity amount corresponding to each state is acquired.

Learning is performed by utilization of k-nearest neighbors algorithm with a known mental state as an objective variable and a unit time muscle activity amount in the mental state acquired from learning data as an explanatory variable. In the present example, two learning models are prepared, a unit time muscle activity amount acquired from variable band data S (unit time area of variable band) being used as an explanatory variable in one thereof, and a unit time muscle activity amount acquired from rectified myoelectric signal data W (unit time area of myoelectric signal) being used as an explanatory variable in the other thereof. At that time, a value (such as 0.05 V·s) having a dimension, which is a dimension of w×a dimension (s) of unit time, is used as a value of a variable band area, and dimensions of values of the explanatory variables are made to be the same. Also, as a value of the objective variable, category values expressing corresponding mental states such as a relaxed state and a concentration state (such as 1: first relaxed state, 2: second relaxed state, 3: first concentration state, and 4: second concentration state) are used. Note that in the present example, only a unit time muscle activity amount (unit time area of each signal) is used as an explanatory variable. However, different kinds of information (such as heart rate, heartbeat interval, brain wave, and value of acceleration sensor, temperature sensor, respiration sensor, or perspiration sensor) can be further used in combination.

As illustrated in FIG. 8, with respect to myoelectric signal data W at a sampling rate 100 Hz, a prediction accuracy rate by a muscle activity amount acquired by utilization of the rectification method is 83.3% on average, and a prediction accuracy rate by a muscle activity amount acquired by utilization of the signal-processing method (band area method) of the present invention (muscle activity amount based on variable band) is 87.5% on average. There is a slight improvement in the signal-processing method of the present invention. On the other hand, when the sampling rate is decreased to 2 Hz, the prediction accuracy rate of when the signal-processing method of the present invention is used keeps accuracy and is 87.5% while the prediction accuracy rate of when the rectification method is used is significantly decreased to 77.1%.

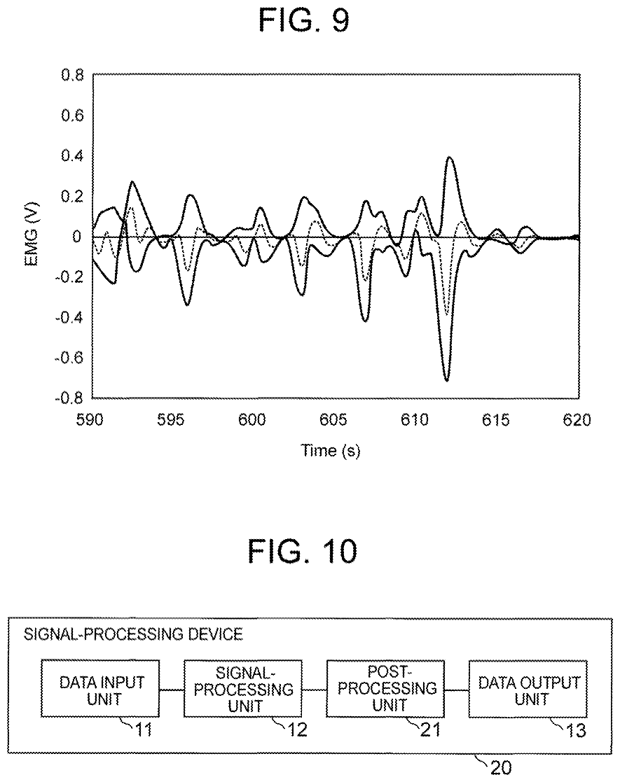

FIG. 9 is a graph illustrating an example of myoelectric signal data W acquired at a sampling rate of 2 Hz and a band (B±k*Σ) acquired therefrom. Note that a broken line is W and a solid line is a band. It is understood that the myoelectric signal data W at the sampling of 2 Hz which data is illustrated in FIG. 9 loses a part of signal components when compared with the myoelectric signal data W at the sampling rate of 100 Hz which data is illustrated in FIG. 7. However, it is understood that the band illustrated in FIG. 9 expresses a change tendency of the myoelectric signal data W at 2 Hz well. This is also understood from the fact that there is no large difference, in a band area in a time interval in which muscle potential is generated, between the case of 100 Hz and the case of 2 Hz.

In the band area method, accuracy of information becomes higher as a sampling rate becomes higher (for example, 100 Hz). However, even when the sampling rate is reduced to some extent (for example, to 2 Hz), accuracy of information extracted from a signal can be kept.

Note that according to the band area method, an effect can be acquired in principle, for example, even when a sampling rate is reduced to twice a reciprocal of a length of data time of when desired information is extracted. For example, when it is assumed that desired information is calculated from data of 180 seconds, there is an effect in principle even when one piece of data is acquired at a sampling rate of 1/90 Hz, that is, in every 90 seconds. Thus, a target of processing of the signal-processing device may be series data in which data is observed in a period equal to or shorter than ⅔ or equal to or shorter than ½ of duration of a time change that is observation target according to the series data.

Such an effect by the band area method is specifically significant for a signal that does not have a clear periodic feature, an incidental signal, a signal characterized by a pulse, and data having a vertical wave motion with respect to a curved baseline.

### Second Exemplary Embodiment

Next, the second exemplary embodiment of the present invention will be described. According to a purpose, various kinds of post-processing can be performed with respect to variable band data S acquired by the above band area method. FIG. 10 is a block diagram illustrating a configuration example of a signal-processing device 20 of the second exemplary embodiment. The signal-processing device 20 illustrated in FIG. 10 is different from the signal-processing device 10 of the first exemplary embodiment illustrated in FIG. 1 in a point of further including a post-processing unit 21.

The post-processing unit 21 performs predetermined post-processing on variable band data S acquired by a signal-processing unit 12, and extracts predetermined information.

The post-processing unit 21 may calculate, for example, a feature amount corresponding to the above muscle activity amount or unit time muscle activity amount, that is, an area or a unit time area in a predetermined time period of a variable band.

Also, for example, the post-processing unit 21 may acquire a feature amount indicating an energy variation tendency of a signal by performing first-order differential processing. Also, for example, the post-processing unit 21 can extract time indicating a variability extreme value (index i) by performing second-order differential processing. In addition, the post-processing unit 21 may detect an abnormal signal in a raw signal (series data W) by using variable band data S. For example, the post-processing unit 21 can perform threshold determination with intensity of the variable band data S as a reference and output a result thereof, and can activate a trigger on the basis of a threshold determination result.

Also, the post-processing unit 21 may detect a place and width of a peak existing in the raw signal on the basis of a place or width of a peak existing in the variable band data S. Also, the post-processing unit 21 can provide a filter function by using these, and can extract only a component of the peak existing in the raw signal, for example.

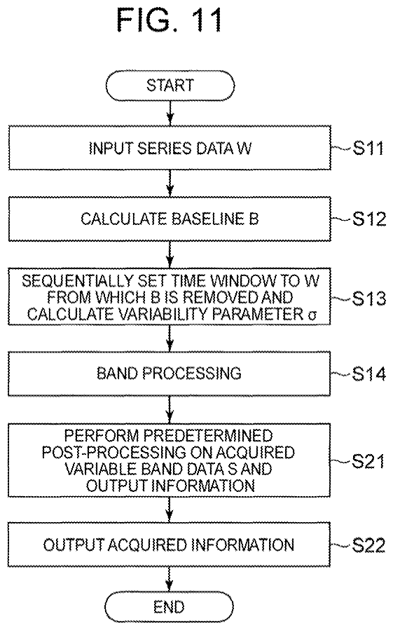

FIG. 11 is a flowchart illustrating an operation example of the signal-processing device 20 of the second exemplary embodiment. In FIG. 11, the same reference sign is given to what is the same as the operation of the signal-processing device 10 of the first exemplary embodiment illustrated in FIG. 3, and a description thereof is omitted. In the present exemplary embodiment, Step S21 to Step S22 are added after band processing in Step S14.

When variable band data S is input from the signal-processing unit 12, the post-processing unit 21 of the signal-processing device 20 performs predetermined post-processing on the variable band data S (Step S21). Then, the post-processing unit 21 outputs information acquired by the post-processing (Step S22).

As described above, according to an information-processing device of the exemplary embodiment, it is possible to extract and output significant information from series data. At that time, it is possible to quickly output significant information from a small amount of series data by sequentially acquiring and processing variable band data S. Also, it is possible to output significant information with a small amount of information compared to series data W by performing threshold determination, statistical processing, or the like on the variable band data S.

### Third Exemplary Embodiment

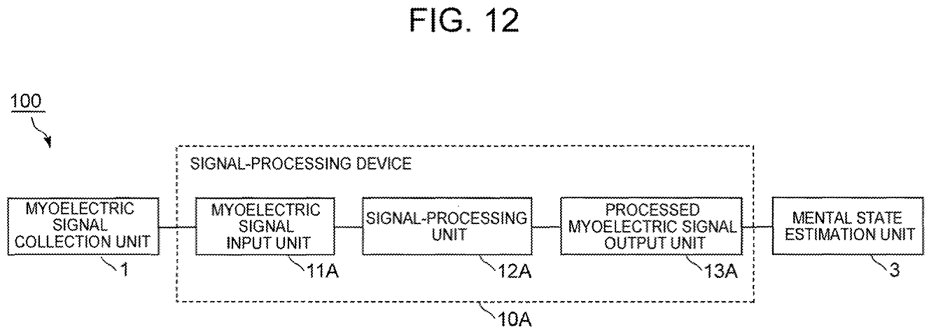

Next, a third exemplary embodiment of the present invention will be described. FIG. 12 is a block diagram illustrating a configuration example of an analysis system of the third exemplary embodiment. The analysis system illustrated in FIG. 12 is an example of usage of the signal-processing device 10 of the first exemplary embodiment.

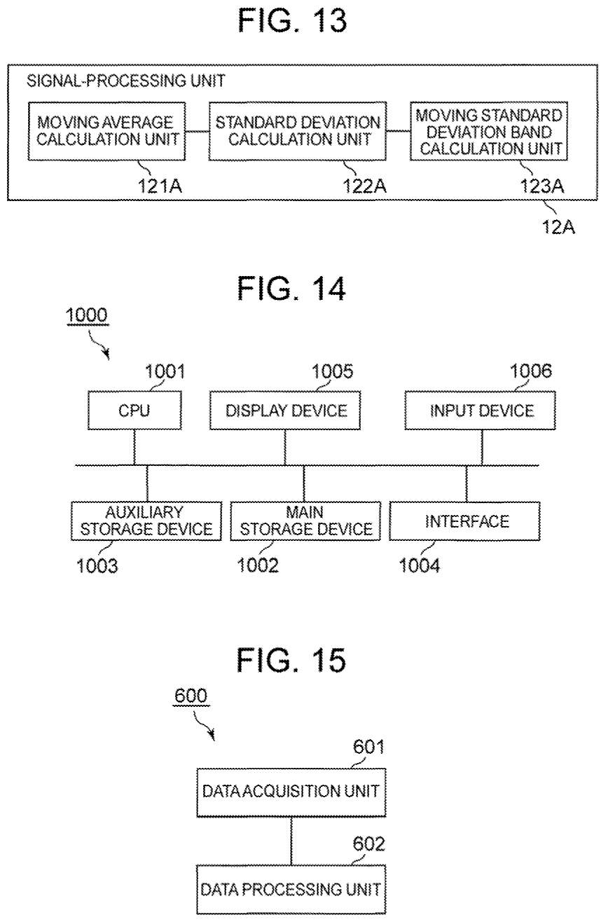

An analysis system 100 illustrated in FIG. 12 includes a myoelectric signal collection unit 1, a signal-processing device 10A, and a mental state estimation unit 3. Also, the signal-processing device 10A includes a myoelectric signal input unit 11A, a signal-processing unit 12A, and a processed myoelectric signal output unit 13A. Also, as illustrated in FIG. 13, the signal-processing unit 12A includes a moving average calculation unit 121A, a standard deviation calculation unit 122A, and a moving standard deviation band calculation unit 123A.

The myoelectric signal collection unit 1 collects (measure) myoelectric signals at a predetermined sampling rate by using an electrode attached near an eye or the like of a person.

The myoelectric signal input unit 11A of the signal-processing device 10A inputs, as series data W, myoelectric signal data that is time-series data of the myoelectric signals collected by the myoelectric signal collection unit 1, and sequentially performs an output thereof to the signal-processing unit 12A in the following stage. Note that in the myoelectric signal data, each myoelectric signal (data w) is associated with an index i corresponding to own collection time.

In the signal-processing unit 12A, first, the moving average calculation unit 121A calculates a moving average with respect to the input myoelectric signal data (W) and acquires a baseline B.

The standard deviation calculation unit 122A sequentially applies a time window TWᵢ to the myoelectric signal data (W) and the baseline B, calculates a standard deviation (σᵢ=Σ(i)) of a variation D of W with respect to B in the time window TWᵢ, and acquires a variability parameter curve Σ.

The moving standard deviation band calculation unit 123A forms a variable band by multiplying a moving standard deviation (Σ(i)), which is each piece of data of the variability parameter curve Σ, by a predetermined constant in a positive/negative direction, calculates a bandwidth thereof in each i of w as variable band data S(i), and acquires variability band data S.

The processed myoelectric signal output unit 13A outputs the variable band data S generated by the signal-processing unit 12A.

The mental state estimation unit 3 estimates a mental state of a person using the variable band data S. For example, with an integration value in a predetermined time length of the variable band data S as a muscle activity amount, the mental state estimation unit 3 estimates the mental state on the basis of the muscle activity amount, or a unit time muscle activity amount in the time period. For the estimation, a prediction model that learns relevance between an explanatory variable previously calculated from the variable band data S and a mental state is used. Note that as already described, it is also possible to use, as an explanatory variable, information other than the information related to the muscle activity amount.

In the analysis system 100, the myoelectric signal collection unit 1 and the signal-processing device 10A may be mounted in a wearable device. Also, a signal-processing device 10A may further include, in the following stage of a signal-processing unit 12A, a post-processing unit 21A that calculates a myoelectric activity amount in a predetermined time length, a unit time muscle activity amount thereof, a moving unit time activity amount associated with i, and the like. In that case, a processed myoelectric signal output unit 13A outputs these pieces of information acquired by the post-processing unit 21A.

(Other)

A signal-processing device of each of the above exemplary embodiments can extract and output significant information from a small amount of data. Thus, for example, when the device is mounted in a device that is a wearable device or the like and that is driven by a battery, an amount of data transmitted by electric power can be controlled, and an effect of improving power saving or a utilization rate of a battery can be further acquired.

Also, according to the signal-processing device of each of the above exemplary embodiments, it becomes possible to extract and output a small amount of significant information from series data. Thus, communication speed can be increased, and utilization for quick feedback is possible. For example, the signal-processing device of each of the above exemplary embodiments can be used in an analysis system that requires high-speed biological information processing.

For example, a human state prediction device used in an automobile driving field or the like requires quick feedback in order to guarantee safe driving even during high-speed driving. Utilization of the signal-processing device of each of the above exemplary embodiments makes it possible to provide such quick feedback.

Also, for example, when the signal-processing device of each of the above exemplary embodiments is applied to a measurement system that is, for example, a radar, a sonar, a lidar (laser radar), or the like and that uses reflection of a pulse wave of light or a sound wave, it is possible to quickly perform peak detection from a small amount of time-series data. Thus, a removal of an effect of external disturbance, an improvement in sensitivity, and scanning in short time become possible.

As a different application example, by using the signal-processing device of each of the above exemplary embodiments in a speech recognition field, it becomes possible to reduce the number of pieces of processing data of a speech and to find a feature or abnormality in the speech at high speed.

Also, for example, when the signal-processing device of each of the above exemplary embodiments is used for a purpose of action prediction or abnormality detection of a person from a moving image, it is possible to stochastically acquire a feature of an action of a person even from a moving image recorded at a low frame rate. Thus, it becomes possible to perform accurate prediction or abnormality detection from a small amount of information.

Also, for example, when the signal-processing device of each of the above exemplary embodiments is used in a field of agricultural facility management or the like, it is possible to monitor existence/non-existence of an abnormality even when a sampling rate of measuring a signal such as a level of carbon dioxide or different noxious gas is low. Thus, a processing speed of a system becomes high and growth management of a plant becomes easy.

Also, other application examples include an observation of electromagnetic waves or cosmic rays such as X-rays in an astronomy field, an observation of seismic waves in a geological field, various analyses such as an elemental analysis and a compositional analysis performed with a pulse wave of an ion, electron, or the like in a material engineering field, and the like.

Even in a purpose other than the above, when the signal-processing device of each of the above exemplary embodiments is used for a purpose of processing existing series data, significant information can be acquired at high speed. Thus, a degree of freedom in operation can be increased.

Also, FIG. 14 is a schematic block diagram illustrating a configuration example of a computer according to each exemplary embodiment of the present invention. A computer 1000 includes a CPU 1001, a main storage device 1002, an auxiliary storage device 1003, an interface 1004, a display device 1005, and an input device 1006.

A device (including processing unit) included in a signal-processing device or an analysis system of each of the above exemplary embodiments may be mounted in the computer 1000. In that case, an operation of each device may be stored in the auxiliary storage device 1003 in a form of a program. The CPU 1001 reads the program from the auxiliary storage device 1003, expands the program in the main storage device 1002, and performs predetermined processing in each exemplary embodiment according to the program. Note that the CPU 1001 is an example of an information-processing device that operates according to the program. In addition to the central processing unit (CPU), a micro processing unit (MPU), a memory control unit (MCU), a graphics processing unit (GPU), or the like may be included, for example.

The auxiliary storage device 1003 is an example of a non-transitory tangible medium. Other examples of a non-transitory tangible medium include a magnetic disk, a magneto-optical disk, a CD-ROM, a DVD-ROM, a semiconductor memory, and the like connected through the interface 1004. Also, in a case where this program is distributed to the computer 1000 through a communication line, the computer 1000 that receives the distribution may expand the program in the main storage device 1002 and execute predetermined processing in each exemplary embodiment.

Also, the program may be for realizing a part of predetermined processing in each exemplary embodiment. Moreover, the program may be a difference program that realizes predetermined processing in each exemplary embodiment in combination with a different program already stored in the auxiliary storage device 1003.

The interface 1004 transmits/receives information to/from a different device. Also, the display device 1005 presents information to a user. Also, the input device 1006 receives an input of information from the user.

Also, depending on processing contents in an exemplary embodiment, a part of elements of the computer 1000 can be omitted. For example, when the computer 1000 does not present information to a user, the display device 1005 can be omitted. For example, when the computer 1000 does not receive an information input from a user, the input device 1006 can be omitted.

Also, a part or whole of each component of each of the above exemplary embodiments is performed by general-purpose or dedicated circuitry, processor, or the like or a combination thereof. These may be configured by a single chip or may be configured by a plurality of chips connected through a bus. Also, a part or whole of each component of each of the above exemplary embodiments may be realized by a combination of the above-described circuitry or the like with the program.

In a case where a part or whole of each component of each of the above exemplary embodiments is realized by a plurality of information-processing devices, circuitry, and the like, the plurality of information-processing devices, circuitry, and the like may be collectively arranged or dispersedly arranged. For example, the information-processing devices, circuitry, and the like may be realized in a form of being connected through a communication network, the form being a client and server system or a cloud computing system, for example.

Next, an outline of the present invention will be described. FIG. 15 is a block diagram illustrating an outline of a signal-processing device of the present invention. A signal-processing device 600 illustrated in FIG. 15 includes a data acquisition unit 601 and a data-processing unit 602.

The data acquisition unit 601 (such as data input unit 11) acquires series data or data included in the series data.

The data-processing unit 602 (such as signal-processing unit 12) generates variable band data that is data related to a bandwidth of a variable band expressed by a predetermined constant multiple in a positive/negative direction of a variability parameter that is a parameter acquired by application of a time window of a predetermined time length to target series data, which is the acquired series data or series data including the acquired data, after a removal of a moving average and that is a parameter indicating variability of the target series data in the time window.

With a configuration in the above manner, even when series data is acquired under an acquisition condition in which a part of valid components is lost, significant information can be acquired from the series data highly accurately.

Although the present invention has been described with reference to the present exemplary embodiment and example, the present invention is not limited to the above exemplary embodiment and example. Various modifications that can be understood by those skilled in the art can be made within the scope of the present invention with respect to a configuration or a detail of the present invention.

Although the present invention has been described with reference to exemplary embodiments, the present invention is not limited to the above exemplary embodiments. Various modifications that can be understood by those skilled in the art can be made within the scope of the present invention with respect to a configuration or a detail of the present invention.

This application claims priority based on Japanese Patent Application No. 2017-253457 filed on Dec. 28, 2017, the entire disclosure of which is incorporated herein.

### INDUSTRIAL APPLICABILITY

The present invention can be specifically suitably applied to series data in which pieces of data are arranged at certain intervals on a predetermined axis and which is series data of signals with a feature of a change appearing in a total amount in a predetermined time length. The signals are, for example, a biological signal, a signal in which a feature of change appears as a pulse, a signal that does not have a clear periodic feature, a signal that changes incidentally, and the like.

### REFERENCE SIGNS LIST

10, 20 Signal-processing device
11 Data input unit
12 Signal-processing unit
121 Baseline calculation unit
122 Variability parameter calculation unit
123 Band processing unit
13 Data output unit
21 Post-processing unit
100 Analysis system
1 Myoelectric signal collection unit
10A Signal-processing device
11A Myoelectric signal input unit
12A Signal-processing unit
121A Moving average calculation unit
122A Standard deviation calculation unit
123A Moving standard deviation band calculation unit
13A Processed myoelectric signal output unit
21A Post-processing unit
3 Mental state estimation unit
1000 Computer
1001 CPU
1002 Main storage device
1003 Auxiliary storage device
1004 Interface
1005 Display device
1006 Input device
600 Signal-processing device
601 Data acquisition unit
602 Data-processing unit

What is claimed is:

1. A signal-processing device comprising:
a data acquisition unit that acquires series data or data included therein; and
a data-processing unit that generates variable band data that is data related to a bandwidth of a variable band expressed by a predetermined constant multiple in a positive/negative direction of a variability parameter that is a parameter acquired by application of a time window of a predetermined time length to target series data, which is the acquired series data or series data including the acquired data, after a removal of a moving average and that is a parameter indicating variability of the target series data in the time window.

2. The signal-processing device according to claim 1, wherein
the data-processing unit performs application of the time window while moving the time window by one piece of data on a time axis of the target series data, calculates the variability parameter in association with an index of the target series data which index is set as a reference of each time window, and generates variable band data indicating a bandwidth, in each time unit of the target series data, of the variable band acquired by multiplication of each of the acquired variability parameters by a predetermined constant in a positive/negative direction while keeping coincidence with the target series data on the time axis.

3. The signal-processing device according to claim 1, wherein
the variability parameter is expressed by a standard deviation of the target series data, from which the moving average is removed, in the time window.

4. The signal-processing device according to claim 1, wherein,
data included in the target series data is a biological signal, a signal in which a feature of a change appears as a pulse, a signal that does not have a clear periodic feature, or a signal that changes incidentally.

5. The signal-processing device according to claim 1, wherein,
a collection period of data included in the target series data is equal to or shorter than ⅔ of duration of a time change that is an observation target according to the data.

6. The signal-processing device according to claim 5, further comprising
a post-processing unit that performs predetermined post-processing by using the variable band data as information indicating a total amount of a signal after rectification which signal is indicated by the data of the target series data.

7. An analysis system comprising:
a signal collection unit that collects a signal at a predetermined sampling rate;
the signal-processing device according to claim 1, in which data included in series data is the signal collected by the signal collection unit; and
a state estimation unit that estimates a state of an observation source of the series data on the basis of information acquired by the signal-processing device.

8. The analysis system according to claim 7, wherein
the signal collection unit collects a myoelectric signal having a correlation with a mental state, and
the state estimation unit calculates, by using variable band data acquired from the signal-processing device as information indicating a total amount of the myoelectric signal after rectification, a muscle activity amount or a unit time muscle activity amount in a predetermined time interval from the variable band data, and estimates a mental state or detects a switch of the mental state of a person who is a collection source of the myoelectric signal on the basis of the calculated activity amount or unit time muscle activity amount.

9. A signal-processing method comprising:
generating, when acquiring series data or data included therein, variable band data that is data related to a bandwidth of a variable band expressed by a predetermined constant multiple in a positive/negative direction of a variability parameter that is a parameter acquired by application of a time window of a predetermined time length to target series data, which is the acquired series data or series data including the acquired data, after a removal of a moving average and that is a parameter indicating variability of the target series data in the time window,
the generating being performed by an information-processing device.

10. A non-transitory computer-readable recording medium in which a signal-processing program is recorded, the signal-processing program causing a computer to execute
processing of acquiring series data or data included therein, and
processing of generating variable band data that is data related to a bandwidth of a variable band expressed by a predetermined constant multiple in a positive/negative direction of a variability parameter that is a parameter acquired by application of a time window of a predetermined time length to target series data, which is the acquired series data or series data including the acquired data, after a removal of a moving average and that is a parameter indicating variability of the target series data in the time window.
